SEQUENCE LISTING (9 sequences)

SEQ ID NO: 1 - protein, 17 aa, Artificial Sequence
  SPIKELMX1 aa positions 812 - 828
PSKRSFIEDLLFNKVTL

SEQ ID NO: 2 - protein, 17 aa, Artificial Sequence
  NCOA1LXD1 aa positions 627 - 643
SQTSHKLVQLLTTTAEQ

SEQ ID NO: 3 - protein, 17 aa, Artificial Sequence
  NCOA2LDX1 aa positions 635 - 651
SKGQTKLLQLLTTKSDQ

SEQ ID NO: 4 - protein, 17 aa, Artificial Sequence
  NCOA1LXD2 aa positions 684 - 700
TERHKILHRLLQEGSPS

SEQ ID NO: 5 - protein, 17 aa, Artificial Sequence
  NCOA2LDX2 aa positions 684 - 700
KEKHKILHRLLQDSSSP

SEQ ID NO: 6 - protein, 17 aa, Artificial Sequence
  NCOA1LXD3 aa positions 743 - 759
SKDHQLLRYLLDKDEKD

SEQ ID NO: 7 - protein, 17 aa, Artificial Sequence
  NCOA2LDX3 aa positions 739 - 755
KKENALLRYLLDKDDTK

SEQ ID NO: 8 - protein, 17 aa, Artificial Sequence
  NCOA1LXD4 aa positions 907 - 923
QCISSQLDELLCPPTTV

SEQ ID NO: 9 - protein, 17 aa, Artificial Sequence
  NCOA2LDX4 aa positions 1073 - 1089
PSDEGALLDQLYLALRN

(19) United States
(12) Patent Application Publication
(10) Pub. No.: US 2024/0180889 A1
(43) Pub. Date: Jun. 6, 2024

(54) COMBINATION THERAPY FOR COVID-19 VACCINATION

(71) Applicant: Dompe' farmaceutici SpA, Milano (IT)

(72) Inventors: Marcello ALLEGRETTI, L'Aquila (IT); Andrea Rosario BECCARI, Napoli (IT); Maria Candida CESTA, L'Aquila (IT); Elisabetta Maria Ester MAURI, Milano (IT); Carmine TALARICO, Napoli (IT)

(21) Appl. No.: 18/284,951

(22) PCT Filed: Mar. 31, 2022
(86) PCT No.: PCT/EP2022/058688

§ 371 Date: Sep. 29, 2023

(30) Foreign Application Priority Data
Mar. 31, 2021 (EP) 21166461.0

(51) Int. Cl. A61K 31/4535; A61K 39/00; A61K 39/215

(52) U.S. Cl. CPC A61K 31/4535; A61K 39/215; A61K 2039/53

(57) ABSTRACT

The present invention relates to the combination of a Selective Estrogen Receptor Modulator (SERM) and a COVID-19 vaccine.

### FIELD OF THE INVENTION

The present invention relates to the combination of an estrogen receptor modulator with COVID-19 vaccination.

### STATE OF THE ART

Coronaviruses (Covs) are a large family of single-stranded, enveloped RNA viruses that belong to the Coronaviridae family. The limited number of coronaviruses known to be able to infect humans were considered in the past as relatively harmless respiratory human pathogens, causing mild infections. However, two coronavirus subtypes have emerged, Severe Acute Respiratory Syndrome Coronavirus (SARS-COV) and the Middle East Respiratory Syndrome Coronavirus (MERS-COV), that cause severe and sometimes fatal respiratory tract infections in humans (Pereira, H. G., 1989 Coronaviridae, In J. S. Porterfield (ed.), Andrewes' Viruses of Vertebrates, 5th ed.pp. 42-57; Holmes K. V. et al., Fields Virology 1996, 1: 1075-1093). In December 2019, atypical pneumonia cases occurred in China and the cause was later identified as being a novel coronavirus. The World Health Organization (WHO) named the virus as SARS-COV-2 and the related disease as COVID-19.

The virus spread rapidly worldwide, and on 11 Mar. 2020 the WHO declared SARS-CoV-2 infection as a pandemic. Most people infected with COVID-19 experience mild to moderate respiratory illness (fever, fatigue, dry cough and dyspnea) and recover without requiring special treatments. Older people, and those with underlying medical problems like cardiovascular disease, diabetes, chronic respiratory disease, and cancer are more likely to develop serious illness. Furthermore, analysis of epidemiological and clinical characteristics and outcomes of patients infected by SARS-COV-2 have shown that 15% of people with symptomatic COVID-19 have significant disease including severe pneumonia, and 5% experience critical disease with life-threatening complications. Critical disease includes acute respiratory distress syndrome (ARDS), sepsis, septic shock, cardiac disease, thromboembolic events, such as pulmonary embolism and multi-organ failure.

There is also growing evidence that in those who develop critical COVID-19 disease, long-term consequences such as rare neurological and psychiatric complications are reasonably expected. These may include stroke, delirium, anxiety, depression, damage or inflammation of the brain and sleep disturbances.

Due to the dramatic rise in cases and deaths worldwide and the social and economic consequences thereof, efforts have focused on the development of safe and effective vaccines to help control spreading of the disease and bring to an end the COVID-19 pandemic.

Different vaccines are currently under development and some have already been approved for the prevention of COVID-19.

All COVID-19 vaccines and vaccine candidates act by inducing an immune response against virus protein antigens.

SARS-COV-2 is characterised by four structural proteins, namely spike (S) protein, envelope (E) protein, membrane (M) protein and nucleocapsid (N) protein (Chen Y et al, J Med Virol 2020, 92: 418-423).

Currently, the spike (S) protein is used as a target antigen in COVID-19 vaccines. This protein is very exposed, since it is located at the surface of the virus, and it is considered an important antigenic determinant capable of inducing a protective immune response (Ou et al, Nat Commun 2020; 11: 1620).

Furthermore, it is an essential molecule for entry of the virus into the cells through the cell entry receptor Angiotensin-converting enzyme II (ACE2). The sequence of the spike protein (UniProt ID: PODTC2) is 1273 aminoacids long and consists of a signal peptide (amino acids 1-13) located at the N-terminus, the S1 subunit (14-685 residues), and the S2 subunit (686-1273 residues); the last two regions are responsible for receptor binding and membrane fusion, respectively. In particular, the S1 subunit mediates receptor binding to ACE2 through the receptor binding domain (RBD) and the S2 subunit is responsible for membrane fusion (Letko et al, Nat Microbiol 2020, 5: 562-569). The vaccines and vaccine candidates developed or under development for COVID-19 are mainly classified into two broad categories: traditional inactivated or live-attenuated virus vaccines, that introduce viral protein antigens into the body of the host and newer gene-based vaccines, such as DNA vaccines and mRNA vaccines.

DNA and mRNA vaccines deliver genes encoding the viral protein antigens for in vivo production in the cells of the host and are considered the best approach for COVID-19 vaccination compared to the traditional vaccine approaches, in view of their improved safety and suitability for mass manufacturing.

The three vaccines approved so far by EMA are all based on this technological approach: the mRNA based Comirnaty developed by Pfizer and BioNTech, the mRNA based COVID-19 Vaccine developed by Moderna and the DNA, viral vector based COVID-19 ChAd0x1-S vaccine developed by Oxford University and AstraZeneca.

The administration of these vaccines to a subject induces the in situ production in the body of the spike protein, that causes the immune system to initiate an immune response. Recent evidence has suggested a potential activity of the SARS-COV-2 spike protein on cell signalling in addition and independently from its function in facilitation of viral infection. For example, it has been shown that the full length S1 subunit of SARS-COV-2 spike protein can induce pro-inflammatory responses in vitro in murine and human macrophages (Shirato et al, Heyon 2021, 7(2) e06187) and can activate cell signaling events in cultured human vascular cells (Suzuki et al, Vascular Pharmacology 137 (2021) 106823).

In view of the above, it has been suggested that the presence in the body of the spike protein produced by vaccination may interfere with biological processes, thereby hindering the immune response and/or causing side effects in the short and long term. Estrogens are a group of steroid hormones that play an important role in the normal sexual and reproductive development in women, among which estradiol (E2) is the most potent and prevalent.

These hormones regulate a wide range of physiological functions such as, for example, development and maintenance of secondary sexual characteristics, metabolism, bone homeostasis, blood salt balance, immune and inflammatory responses, responses to stress and/or neuronal function.

An unbalance in the levels or activity of these hormones has been implicated in many pathological processes. In particular, estrogens have been implicated in the etiology of breast, endometrial, renal and uterine cancers (Okamoto et al, Toxicology Letters 2020, 318: 99-103). Furthermore, a decrease in the levels of estrogens have been associated to large increases in bone resorption and early and late forms of osteoporosis in postmenopausal women (Riggs B. L, J. Clin. Invest 2000, 106, 1203-1204)

Furthermore, administration of exogenous estrogens has been associated to a disturbed regulation of numerous aspects of the hemostatic and fibrinolytic pathways that contribute to the generation of a prothrombotic milieu and to a higher risk of thrombotic events (Abou-Ismail et al, Thrombosis Research 2020, 192: 40-51).

The activity of estrogens is mediated by a nuclear receptor, the estrogen receptor, that functions as a hormone-activated transcription factor. Upon ligand activation, this receptor binds to the promoters of target genes and form a transcriptional complex with a number of interacting proteins, collectively known as coregulators, that can either activate (coactivators (CoA)) or inactivate (corepressors (CoR)) transcriptional activity, thereby triggering the activation or repression of target gene expression (Patel et al, Pharmacology & Therapeutics 2018, 186: 1-24). The interaction between the nuclear estrogen receptor and coactivators is mediated by an LxxLL motif (where L is leucine and x is any amino acid) contained and shared within the coactivator proteins, that is necessary and sufficient for the binding of these proteins to the receptor and for enhancing its transcriptional activity (Patel et al, Pharmacology & Therapeutics 2018, 186: 1-24).

### SUMMARY OF THE INVENTION

The present inventors have surprisingly found that the SARS-COV-2 spike protein interferes with the signalling of the estrogen receptor.

In details, the present inventors have found that the SARS-COV-2 spike protein contains a LxxLL motif that is homolog to that of the nuclear coactivator 1 (NCOA1) that is able to bind to the NCOA1 binding domain on the estrogen receptor and thereby activate its transcriptional activity. The present inventors have also demonostrated in in vitro experiments that SARS-COV-2 spike protein exterts an estrogen-like activity in different experimental settings.

Therefore, in the context of COVID-19 vaccination, the spike protein produced following vaccination may interfere with the activity of the estrogen receptor and create an unbalance between physiological and pathological activities mediated by this receptor, thus generating unwanted, serious side effects. Furthermore, due to the role of estrogen in regulating the immune response, the activation of the estrogen receptor by the spike protein may interfere with the efficacy of the vaccine.

The present inventors have also surprisingly found that the activity of spike on the estrogen receptor can be reverted by the administration of a Selective Estrogen Receptor Modulator (SERM).

Selective Estrogen Receptor Modulators (SERMs) are a group of compounds with non-steroidal structures that bind to the estrogen receptor and modulate its activity. SERMs may act as either agonist or antagonist of ER in a tissue-specific manner. It has been suggested that SERMs exert their activity on estrogen receptor signalling by binding to the receptor and inducing distinct conformational changes that influence its ability to interact with coactivators and corepressors.

In view of the above data, the administration of a SERM in combination with the administration of a COVID-19 vaccine is expected to be beneficial in order to improve the immune response and decrease the side effects of vaccination.

Accordingly, a first object of the invention is a combination of a SERM, preferably selected from tamoxifen, raloxifene, 4-hydroxy tamoxifen, droloxifene, ospemifene, arzoxifene, toremifene and bazedoxifene and a COVID-19 vaccine.

A further object of the invention is the above combination for use in the prevention of COVID-19 in a subject.

A further object of the invention is a SERM for use in combination with a COVID-19 vaccine in the prevention of COVID-19 in a subject.

A further object of the invention is a SERM for use in the prevention or treatment of side effects of a COVID-19 vaccine in a subject.

A further object of the invention is a pharmaceutical composition comprising a SERM and pharmaceutically acceptable excipients, for use in combination with a COVID-19 vaccine in the prevention of COVID-19 in a subject.

A further object of the invention is a pharmaceutical composition comprising a SERM and pharmaceutically acceptable excipients, for use in the prevention of side effects of a COVID-19 vaccine in a subject.

A further object of the invention is a kit of parts comprising:
a) a COVID-19 vaccine; and
b) a pharmaceutical composition comprising an effective amount of a SERM.

A further object of the invention is a method for the prevention of COVID-19 in a subject comprising the administration to said subject of a COVID-19 vaccine in combination with a SERM.

A further object of the invention is a method for the prevention or treatment of side effects of a COVID-19 vaccine in a subject comprising the administration of a SERM to said subject.

### DESCRIPTION OF THE FIGURES

FIG. 1 shows the Estrogen Receptor (ER) complexed with nuclear receptor coactivator. The whole complex is reported in cartoon; the ER monomers are in gray surface, while the NCOA segments are reported in black cartoon and lines.

FIG. 2 shows the network of the most significant interactions of ER1 and ER2 (both in red spheres). Among the most reliable interacting proteins, the Nuclear receptor COActivators (NCOAs) directly bind the nuclear receptors and stimulate transcriptional activities.

FIG. 3 shows the sequence alignment among LDX region in spike (aminoacids 818-822) and other LDX cofactor-receptor domains.

FIG. 4 shows the 3D best docking hypothesis for spike-ER blind docking. The proteins are reported in cartoon. The ER dimer is in white cartoon while the spike protein is reported in black surface.

FIG. 5 shows the 3D best docking hypothesis for spike-ER motif-oriented docking. The proteins are reported in cartoon. The ER dimer is in white cartoon while the spike protein is reported in black surface.

FIG. 6 shows the proliferation of MCF-7 cells treated for 24 hours with estradiol (1 nM) (ESTR), spike protein (10 ng/ml) (SPIKE), raloxifene (2 μM) (RAL), a combination of estradiol (1 nM) and spike protein (10 ng/ml) (ESTR+SPIKE), a combination of estradiol (1 nM) and raloxifene (2 μM) (ESTR+RAL), a combination of spike protein (10 ng/ml) and raloxifene (2M) (SPIKE+RAL), a combination of estradiol (1 nM), spike protein (10 ng/ml) and raloxifene (2 μM) (ESTR+SPIKE+RAL), measured by BrdU proliferation assay as described in Example 5. The experiment was performed in technical triplicate. Results are mean±SD of 3 different experiments (n=3).

FIG. 7 shows the proliferation of MDA-MB-231 cells treated for 24 hours with estradiol (1 nM) (ESTR), spike protein (10 ng/ml) (SPIKE), raloxifene (2 μM) (RAL), a combination of estradiol (1 nM) and spike protein (10 ng/ml) (ESTR+SPIKE), a combination of estradiol (1 nM) and raloxifene (2 μM) (ESTR+RAL), a combination of spike protein (10 ng/ml) and raloxifene (2 μM) (SPIKE+RAL) a combination of estradiol (1 nM), spike protein (10 ng/ml) and raloxifene (2 μM) (ESTR+SPIKE+RAL), measured by BrdU proliferation assay as described in Example 5. The experiment was performed in technical triplicate. Results are mean±SD of 3 different experiments (n=3).

FIG. 8 shows TRAP activity, expressed in U/L, of RAW cells not differentiated to osteoclast and treated with vehicle (CTR w/o RANKL), RAW-OC cells treated for 24 hours with vehicle (CTR with RANKL), 17β-estradiol (1 nM) (Estradiol), spike (10 ng/ml) (Spike), Raloxifene (2 μM) (Raloxifen), a combination of spike (10 ng/ml) and Raloxifene (2 μM) (Spike+Raloxifene), a combination of spike (10 ng/ml) and 17ß-estradiol (1 nM) (Spike+Estradiol), a combination of 17β-estradiol (1 nM) and Raloxifene (2 μM) (Estradiol+Raloxifene), a combination of 17β-estradiol (1 nM), spike (10 ng/ml) and Raloxifene (2 UM) (Estradiol+Spike+Raloxifene), measured as described in Example 6. Data are reported as U/L. The experiment was performed in technical six replicates. Results are mean±SD of one experiment representative of three performed.

### DETAILED DESCRIPTION OF THE INVENTION

A first object of the invention is a combination of a Selective Estrogen Receptor Modulator (SERM) and a COVID-19 vaccine.

As already discussed above, the present inventors have found that the administration of the above combination reduces side effects and improves immune response compared to the administration of the COVID-19 vaccine alone.

Thus, a further object of the present invention is the above combination for use in the prevention of COVID-19 in a subject.

A further object of the invention is a SERM for use in combination with a COVID-19 vaccine in the prevention of COVID-19 in a subject.

A further object of the invention is a SERM for use in the prevention or treatment of side effects of a COVID-19 vaccine in a subject.

Said subject is preferably a human being.

Said COVID-19 vaccine is a COVID-19 spike protein-based vaccine.

With the wording “spike protein-based vaccine” it is meant any vaccine against COVID-19 that uses the spike protein of SARS-COV-2, a variant thereof or an immunogenic fragment thereof as an antigen. This includes any vaccine whose administration to a subject introduces into the body of such subject the spike protein of SARS-COV-2, a variant thereof or an immunogenic fragment thereof either as such or bound to the inactivated or live attenuated virus. Also, this definition includes any mRNA or DNA vaccine that causes the production of the SARS-COV-2 spike protein, a variant thereof or an immunogenic fragment thereof in situ after administration into the human body. Preferably, said COVID-19 vaccine is a COVID-19 mRNA or DNA vaccine.

In the context of the present invention, the wording “COVID-19 mRNA or DNA vaccine” refers to any vaccine that comprises mRNA or DNA whose administration to a subject result in the production into such subject of the SARS-COV-2 spike protein, a variant thereof or an immunogenic fragment thereof.

According to the present invention, by “variant” of SARS-COV-2 spike protein it is meant a protein having an aminoacid sequence which differs from the sequence of SARS-COV-2 spike protein in that it contains one or more substitutions, deletions of internal amminoacids or insertions of one or more aminoacids. Preferably, said substitutions, deletions or insertions maintain the antigenic properties of the SARS-COV-2 spike protein. Preferably, said variant has a sequence with an aminoacid identity with SARS-COV-2 spike protein of at least 90%, 95%, 98%, 99%.

According to the present invention, by “immunogenic fragment” of SARS-COV-2 spike protein it is meant a protein having an aminoacid sequence which correspond to an N-terminally and/or C-terminally truncated sequence of SARS-COV-2 spike protein or of a variant thereof and maintaining the antigenic properties of the SARS-COV-2 spike protein or the variant thereof.

Said immunogenic fragment of the spike protein preferably has an aminoacid sequence consisting of at least 50%, 60%, 70%, 80% or 90% of the aminoacid sequence of the full-length SARS-COV-2 spike protein.

Preferably, said immunogenic fragment contains one or more of the following aminoacids sequences of the full-length SARS-COV-2 spike protein (UniProt ID: PODTC2): from position 232 to 246, from position 233 to 247, from position 471 to 503, from position 604 to 625, from position 817 to 833, from position 818 to 822, from position 891 to 907, from position 897 to 913, from position 1164 to 1191, from position 1182 to 1209.

Said SERM is preferably selected from tamoxifen, raloxifene, 4-hydroxy tamoxifen, droloxifene, ospemifene, arzoxifene, toremifene and bazedoxifene.

A particularly preferred SERM according to the invention is raloxifene.

As discussed above, the use of a SERM in combination with a COVID-19 vaccine according to the invention prevents side effects resulting from the interference of the spike protein produced in the host after administration of the COVID-19 vaccine with the physiological function of the estrogen receptor and improves the response of the immune system to the vaccine.

These findings result in an improved therapy for preventing COVID-19.

Preferably, said side effects are unwanted effects due to an overactivation of the estrogen receptor.

Thus, the administration of the SERM according to the invention is particularly advantageous in case of subjects that may have more serious side effects as a result of administration of the COVID-19 vaccine and the associated increase in the activity of estrogen receptor. These are for example subjects diagnosed with ER-positive breast cancer or with any pathological condition that alter the physiological haemostatic balance such as a blood clotting disorder.

According to one embodiment, said subject is a subject diagnosed with an ER-positive breast cancer.

According to another embodiment, said subject is a subject diagnosed with a pathological condition that alter the physiological haemostatic balance.

The administration of the SERM in combination with the COVID-19 vaccine according to the invention is also advantageous to all other categories of subjects in order to avoid side effects and improve immune response to the COVID 19 vaccine.

Therefore, according to one embodiment, said subject is a subject that has not been diagnosed with an ER-positive breast cancer.

According to another embodiment, said subject is a subject that has not been diagnosed with a pathological condition that alter the physiological haemostatic balance.

According to another embodiment, said subject is a subject that has not been diagnosed with an ER-positive breast cancer, a pathological condition that alter the physiological haemostatic balance.

In accordance with all the objects of the invention, the SERM can be administered concurrently or separately from the COVID-19 vaccine.

Preferably, it is administered separately from the COVID-19 vaccine and with a different schedule of administration.

Preferably, the SERM is administered to the subject having received, receiving or due to receive a COVID-19 vaccine, preferably once or twice a day, for a period of time starting SERMtwo weeks and the day of administration of the first dose of vaccine and ending at least two weeks after the administration of the last dose of vaccine, more preferably at least one month after the administration of the last dose of vaccine.

The frequency of administration and duration of the treatment will vary depending on the dose of SERM used and/or on the type of COVID-19 vaccine.

According to one embodiment, the SERM is used at the dose and frequency at which it is usually employed for the treatment of other pathologies.

More preferably, in order to determine the correct dose and schedule of administration, the levels of estrogens in the subject due to receive the COVID-19 vaccine is measured before vaccination and the treatment with the SERM planned on the basis of their basal levels in the patient.

The SERM is preferably administered to the subject having received, receiving or due to receive the COVID-19 vaccine in form of a pharmaceutical composition.

Accordingly, a further object of the present invention is a pharmaceutical composition comprising a SERM, as previously described, and pharmaceutically acceptable excipients, for use in combination with a COVID-19 vaccine for the prevention of COVID-19 in a subject, as previously described.

A further object of the invention is a pharmaceutical composition comprising a SERM, as previously described and pharmaceutically acceptable excipients, for use in the prevention or treartment of side effects of a COVID-19 vaccine in a subject, as previously described.

Preferably, the pharmaceutical composition of the present invention is prepared in suitable dosage forms comprising an effective amount of SERM and pharmaceutically acceptable excipients.

The administration of the pharmaceutical composition of the present invention to a subject is in accordance with known methods and may comprise oral administration, parenteral administration, preferably selected from intravenous, intraperitoneal, intramuscular, intraarterial, subcutaneous administration, topical administration, buccal administration or rectal (suppository) administration.

According to the present invention, the wording “effective amount” means a dosage of a compound or composition sufficient to significantly achieve the desired response.

The vaccine and SERM may also be present as a kit of parts.

Accordingly, another object of the present invention is a kit of parts comprising:
a) the COVID-19 vaccine as described above, and
b) a pharmaceutical composition comprising a SERM, as described above.

The route of administration of the SERM or pharmaceutical composition for use according to the present invention depends on the specific compound used or contained in the pharmaceutical composition and is in accordance with known methods for administration of the compound, which is usually by systemic administration, preferably by oral, parenteral or inhalatory route. The term parenteral as used herein includes intravenous, intraperitoneal, intracerebral, intrathecal, intracranial, intramuscular, intraarticular, intrasynovial, intrasternal, intraocular, intraarterial, subcutaneous, intracutaneous injection or infusion techniques.

The composition of the present invention may be formulated into oral, inhalatory or injectable dosage forms such as tablets, capsules, powders, solutions, suspensions, and emulsions.

The pharmaceutically acceptable excipient according to the present invention includes any and all solvents, diluents, or other vehicle, dispersion or suspension aids, surface active agents, isotonic agents, thickening or emulsifying agents, preservatives, solid binders, lubricants and the like, as suited to the particular dosage form desired. Some examples of materials which can serve as pharmaceutically acceptable excipient include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; sterilized water; Ringer's solution; buffered saline; dextrose solution; maltodextrin solution; ethyl alcohol; and phosphate buffer solutions. Moreover, the composition of the present invention may be formulated into inhalable or injectable dosage forms such as solutions, suspensions, and emulsions by further adding diluents, dispersants, and surfactants.

Further, the composition of the present invention may be suitably formulated using appropriate methods known in the art or by the method disclosed in Remington's Pharmaceutical Science (recent edition), Mack Publishing Company, Easton Pa. The terms “pharmaceutically acceptable” is intended to define, without any limitation, any material suitable for preparing a pharmaceutical composition to be administered to a living being.

The dosage forms can also contain other traditional ingredients such as: preservatives, stabilizers, surfactants, buffers, osmotic regulators, emulsifiers, sweeteners, colorants, flavourings and the like.

The dosage forms of the pharmaceutical composition of the present invention can be prepared by techniques that are familiar to a pharmaceutical chemist, and comprise mixing, granulation, compression, dissolution, sterilization and the like.

A further object of the invention is a method for the prevention of COVID-19 in a subject comprising the administration to said subject a COVID-19 vaccine in combination with a SERM.

A further object of the invention is a method for the prevention or treatment of side effects of a COVID-19 vaccine in a subject comprising the administration to said subject of a SERM.

### EXPERIMENTAL PART

### Examples 1-4

### Materials and Methods

### 1. Interactome Analysis

The STRING database [Szklarczyk D et al., Nucleic Acids Res. 2021, 49: D605-D612, doi: 10.1093/nar/gkaa1074], that integrate all known and predicted associations between proteins, including both physical interactions as well as functional associations was used to analyse functional associations between biomolecules. Each protein-protein interaction is annotated with a ‘score’. This score does not indicate the strength or the specificity of the interaction, but only the confidence. All scores rank from 0 to 1, with 1 being the highest possible confidence.

### 2. 3D Model Selection

Spike 3D model was built based on PDB 6VYB returned to its wild-type form and fully glycosylated. An asymmetric glycosylation of the three protomers was derived by glycoanalyitic data for the N-glycans and O-glycans as published (Casalini L et al, ACS Cent. Sci. 2020, 6:1722-1734, https://doi.org/10.1021/acscentsci.0c01056). The proteins were modeled using Amber14SB force field (Maier J A et al, J. Chem. Theory Comput. 2015, 11:3696-3713, https://doi.org/10.1021/acs.jctc.5b00255) and the carbohydrate moieties by the GLYCAM06j-1 version of GLYCAM06 force field (Kirschner K N et al, J. Comput. Chem. 2008, 29:622-655, https://doi.org/10.1002/jcc.20820). The so prepared structure was used as starting point for Molecular Docking simulations. Topology files were generated with the pdb2gmx GROMACS tool, using the amber99sb forcefield (Lindorff-Larsen K al, et Proteins 2010,78:1950-1958, https://dx.doi.org/10.1002/prot.22711). Protein was inserted in a triclinic box, extending up to 15 Å from the solute, and immersed in TIP3P water molecules (Jorgensen W L et al, J Chem Phys 1983, 79:926-935, https://dx.doi.org/10.1063/1.445869). Counter ions were added to neutralize the overall charge with the genion GROMACS tool. After energy minimizations, the system was relaxed for 5 ns by applying positional restraints of 1000 KJ mol-1 nm-2 to the protein atoms. Following this step, unrestrained MD simulation was carried out for a length of 1 microsecond, with a time step of 2 fs, using GROMACS 2018.3 simulation package (supercomputer Galileo and Marconi-100, CINECA, Bologna, Italy) (Abraham M J et al., SoftwareX 2015, 1:19-25, https://dx.doi.org/10.1016/j.softx.2015.06.001). V-rescale temperature coupling was employed to keep the temperature constant at 300 K (Bussi G et al, J Chem Phys, 2007 126:014101. https://dx.doi.org/10.1063/1.2408420). The Particle-Mesh Ewald method was used for the treatment of the long-range electrostatic interactions (Darden T et al., J. Chem. Phys 1993, 98:10089, https://doi.org/10.1063/1.464397). The first 5 ns of each trajectory were excluded from the analysis. The trajectory obtained after 1 microsecond MD simulation was clustered in order to obtain representative structures. In particular, the structure used for the docking studies is the first centroid of the first cluster extracted from the MD experiment.

For the estrogen receptor, the XRAY PDB model with code 3OLL was used, containing Estradiol and Nuclear receptor coactivator 1 (Mocklinghoff S et al, ChemBioChem 2010, 11:2251-2254, https://doi.org/10.1002/cbic.201000532).

### 3. Protein-Protein Docking Procedure

The input of two individual proteins, one for the receptor and the other for the ligand, were provided. In particular, the Spike protein and ER were used as receptor and ligand, respectively. Then, the HDOCK tool performs docking to sample putative binding modes through an FFT-based search method, and then scoring the protein-protein interactions. Finally, the top 100 predicted complex structures were provided, and the best ten hypotheses were visually inspected to confirm the reliability of the calculation. The entire workflow is described (Yan n Y et al, Nat Protoc 2020, 15:1829-1852, https://doi.org/10.1038/s41596-020-0312-x).

Given the lack of structural information on the binding mechanism between the Spike protein and the nuclear estrogen receptor (ER), the first check involved the identification of proteins known to interact with estrogen receptors. After identifying these ER interacting proteins, the second step involved the study of any sequence analogies between the spike protein and the ER effector proteins.

### Example 1

### Nuclear Receptor Coactivators and LxxLL Motif

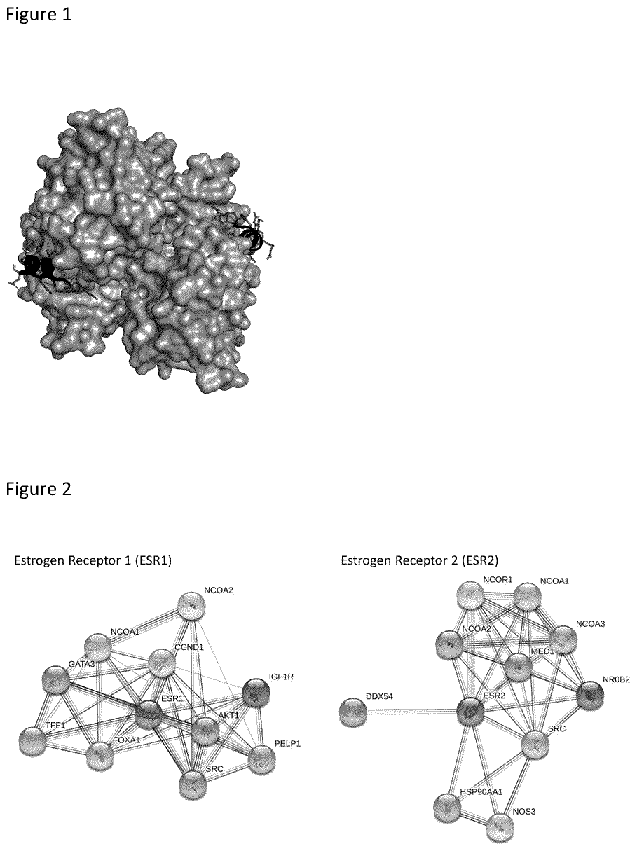

Many transcription factors and co-factors exhibit a common structural motif that ensures interaction with effector proteins. The motif that participates in these protein-protein interactions is termed LxxLL (LDX), and it is associated with different aspects of transcriptional regulation (Plevin M J et al, Trends Biochem Sci 2005, 30:66-69. https://dx.doi.org/10.1016/j.tibs.2004.12.001). The LxxLL sequence was originally identified in proteins that bind the activation function-2 (AF-2) region of nuclear receptor ligand-binding domains (LBDs). These motifs are fundamentals in nuclear-receptor regulation with many nuclear-receptor-binding proteins, including co-activators (NCOA-1, 2 and 3) (Heery D M et al, Nature 1997, 387:733-736, https://doi.org/10.1038/42750). The experimental and structural confirmations which show the interaction between ER and NCOAs, made it possible to focus on the LxxLL motif and starting from this, a mapping of the spike protein sequence was carried out searching for structural motifs and homologous portions of spike able to mimic the interaction between the ER and its nuclear co-activators (FIG. 1).

### Example 2

### Estrogen Receptor Binding Protein Network Analysis

The network of most significant interactions of ER1 and ER2 (both in the spheres) (FIG. 2) highlights that, among the most reliable interacting proteins with ER1 and ER2, the Nuclear receptor COActivators (NCOAs) directly bind nuclear receptors and stimulate transcriptional activities in a hormone-dependent fashion. Many experimental data of complexes between ER and NCOAs deposited in the Protein Data Bank, support these findings, and can be found in the Uniprot database (ER1:
https://www.uniprot.org/uniprot/P03372 #structure; ER2:
https://www.uniprot.org/uniprot/Q92731 #structure).

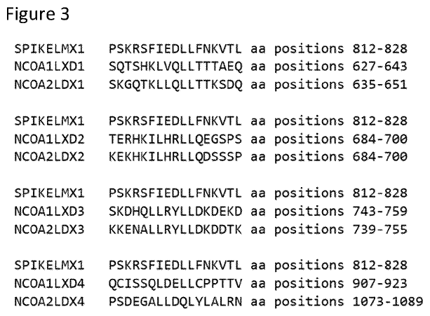

Combining the sequence alignment between NACOs and spike with a three-dimensional analysis of the viral protein, it emerged that the spike protein contains a sequence homologous to LxxLL, also valid from a structural point of view (in fact, this region assumes alpha-helix conformations), in an outer zone and which could, in principle, act as interacting region site with ER (FIG. 3, aminoacids 818 to 828 of SARS-COV-2 spike proteins). Different LDX domain are associated to specific cofactor-receptor interaction and the spike LDX1 resemble NCOA1 LDX4 and belong to Class III domain (Savkur R S et al, J Pept Res 2004, 63:207-212, https://dx.doi.org/10.1111/j.1399-3011.2004.00126.x)

### Example 3

### Spike Protein-ER Blind Docking

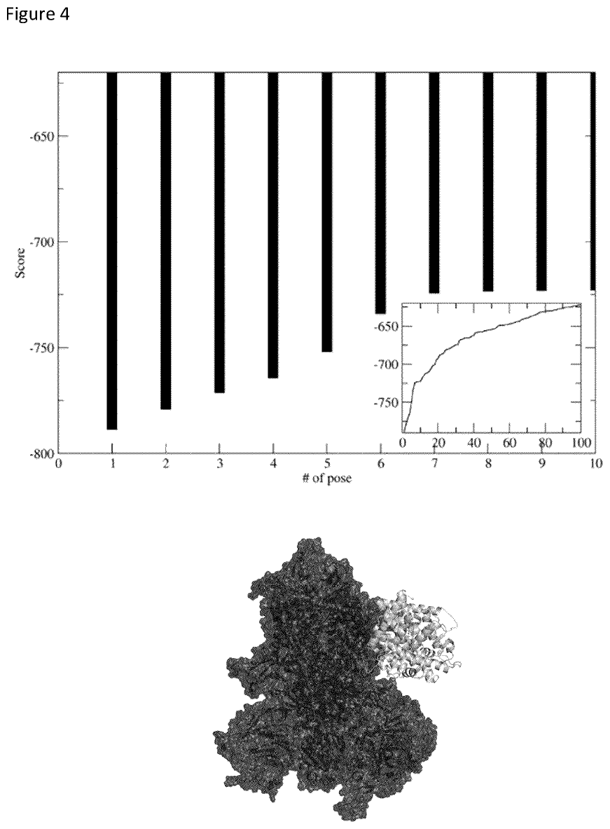

It has been experimentally verified that ER does not bind the Receptor Binding Domain (RBD) of the viral protein (data not shown). To confirm and validate the in silico prediction, the ability of ER to interact in a region other than the viral RBD was evaluated by means of a blind-docking between the two proteins, using the HDOCK server [http://hdock.phys.hust.edu.cn/]. The blind-docking approach does not consider any structural bias and is fully unguided. The best binding hypothesis found puts in evidence a high affinity of ER towards the lateral region of the spike protein, belonging to the so called “fusion peptide portion” (FIG. 4).

### Example 4

### Spike Protein-ER Motif-Oriented Docking

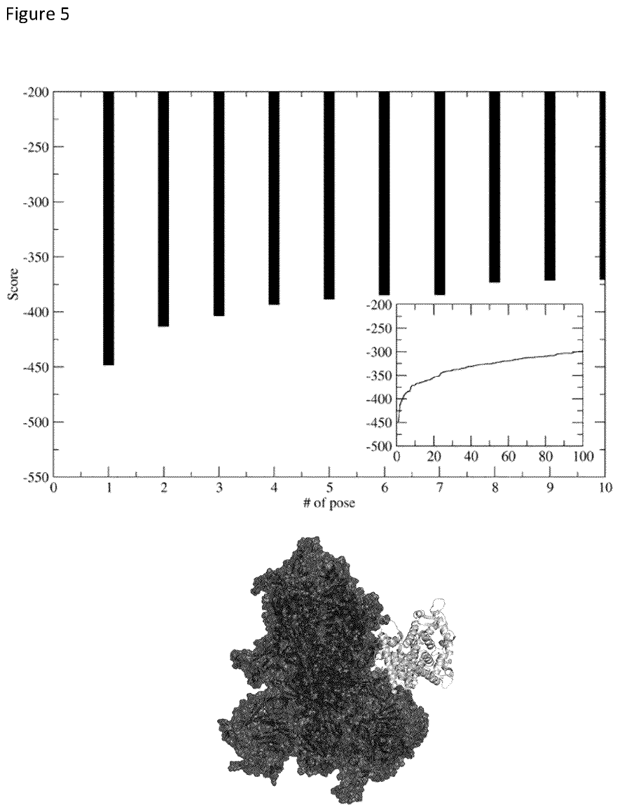

The structural information that ER residues are recognized by NCOAs was used to guide the docking study of ER on spike by optimizing protein-protein interactions. With the protein structures and residue restraints as input, a suitable model was generated by the HDOCK server. Given that the structural information of interaction between ER and NCOA are known, a second docking study was carried out, that considered the ER residues that guarantee interaction with the NCOAs, thus guiding the molecular docking procedure. The best binding hypothesis obtained from the guided docking study is shown in FIG. 5 that highlights the binding of the ER to the spike region containing the motifs homologous to the LxxLL pattern (FIG. 5).

### Example 5

### Induction of Proliferation of Cancer Cells

In this experiment MCF-7 and MDA-MB-231 cells were used. MCF-7 is an invasive ductal/breast carcinoma hormone-dependent cell line (both estrogen and progesterone receptor-positive) and MDA-MB-231 is an epithelial, human breast cancer cell line that lacks the estrogen receptor.

The cells were obtained from ATCC and grew in DMEM without phenol red, supplemented with 10% fetal bovine serum (FBS), penicillin/streptomycin at 37° C. in a 5% CO2 and 95% humidified atmosphere. For each assay cells were seeded at the density of 10⁴ cells/cm². Before treatments, to reduce estrogen levels in FBS and avoiding any interference, cells were cultured for 24h in medium containing 5% dextran-coated charcoal treated serum.

Then, the cells were treated for 24 h with estradiol (1 nM) (ESTR), spike protein (10 ng/ml) (SPIKE), raloxifene (2 μM) (RAL), a combination of 17ß-estradiol (1 nM) and spike protein (10 ng/ml) (ESTR+SPIKE), a combination of 17ß-estradiol (1 nM) and raloxifene (2 μM) (ESTR+RAL), a combination of spike protein (10 ng/ml) and raloxifene (2 μM) (SPIKE+RAL), a combination of 17ß-estradiol (1 nM), spike protein (10 ng/ml) and raloxifene (2 μM) (ESTR+SPIKE+RAL).

A BrdU Proliferation assay was then carried out in oder to measure proliferation of the cells. This assay involves the incorporation of BrdU into the DNA of dividing cells cultured in microtiter plates using the cell layer as the solid phase. BrdU Cell Proliferation ELISA Kit has been used (Abcam, ab126556) following the manufacturer's instructions. BrdU has been added to wells for 24 h and then cells were fixed using Fixing Solution. Then, cells were washed and were incubated with detector anti-BrdU antibody for 1 hour at RT. After incubation, cells were washed and incubated with the horseradish peroxidase conjugated goat anti-mouse antibody for 30 minutes at RT. For the detection the chromogenic substrate tetra-methylbenzidine (TMB) was added and the colored product has been detected using a spectrophotometer (450/550 nm).

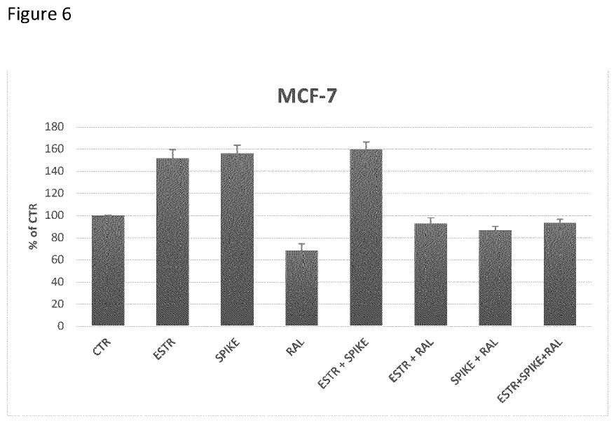

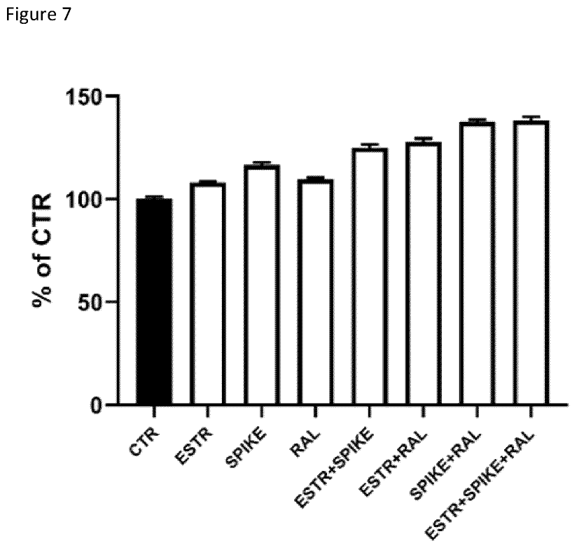

The result obtained are shown in FIG. 6 for MCF7 cells and in FIG. 7 for MDA-MB-231 cells. As can be seen, the SARS-COV-2 spike protein shows a proliferation inducing activity on breast cancer cells that is dependent on the presence of the estrogen receptor. In fact, in MCF-7 cells treatment with 17β-estradiol or spike results in a similar increase in proliferation of the cells while in MDA-MB-231 cells 17β-estradiol and spike, both alone and in combination, do not significantly affect cell proliferation. The proliferation promoting activity of both 17β-estradiol and spike is blocked by treatment with the SERM Raloxifen. These results clearly demonstrate that spike acts on the cells by activating the estrogen receptor and that it is able to induce proliferation of ER positive cancer cells.

### Example 6

### TRAP Activity by ELISA Assay in RAW-OCs

To further demonstrate that spike activates the estrogen receptor, the effect of 17ß-estradiol and SARS-COV-2 spike on TRAP activity was compared in an osteoclast cell model.

The mouse monocytic cell line, RAW 264.7 was used, that differentiate into osteoclasts upon exposure to recombinant RANKL.

In details, RAW264.7 (murine macrophages ATCC, USA) were cultured as manufacturer's protocol. Then 1.5×10⁵ cells/cm² in 24-well dishes were seeded and mouse receptor activator of nuclear factor kb ligand (RANKL, Miltenyi Biotec, Germany) was added at the final concentration of 35 ng/ml to initiate osteoclasts (OC) development (day 0) as previously described (Collin-Osdoby and Osdoby 2012, PMID: 22130930). At day 3, cells were examined under the microscope and refed with fresh medium containing RANKL. At day 6, RAW-OC population was prevalent. Cells were treated with 17-β-estradiol (1 nM), spike (10 ng/ml) and raloxifene (2 μM) and the combination of these for 24h. After 24 h of treatment, the cells were collected in sterile tubes and resuspended in PBS (pH 7.4) to the concentration of approximately 1 million/ml. Then, cells were subjected to repeated freeze-thaw cycles to let out the inside components. In the meantime, the reagents of the kit were brought to room temperature. Tartrate Resistant Acid Phosphatase (TRAP) activity was measured by Enzyme-linked immunosorbent assay purchased by Myobiosource (cat no. MBS1601167). The standard curve, reagents and samples were prepared following manufacturer's protocol. Briefly, 50 μl of standard were added to standard wells and 40 μl of sample-to-sample wells and then added 10 μl of anti-TRAP antibody to sample wells and 50 μl of streptavidin-HRP to sample wells and standard wells. The plate was covered with a sealer, mixed well on a rocking platform, and incubated 1 hour at 37° C. The plate was washed 5 times with wash buffer and 50 μl of substrate solution A were added to each well plus 50 μl of substrate solution B and incubated 10 minutes at 37° C. in the dark. Finally, 50 μl of stop solution to each well were added and the optical density was immediately determined using a microplate reader set at 450 nm.

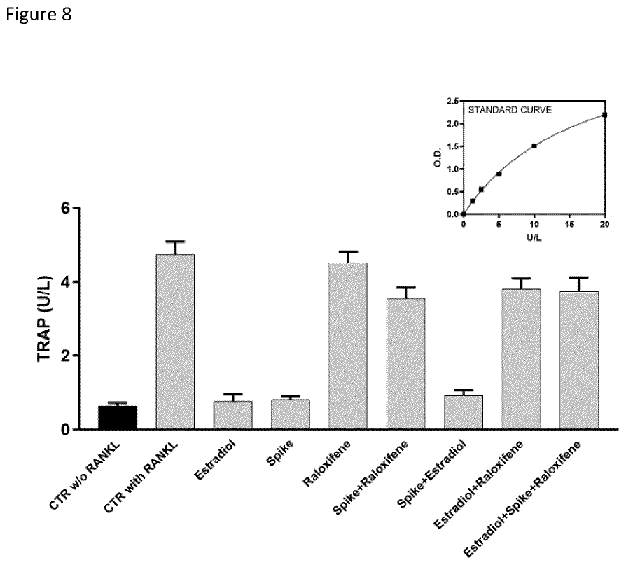

The results obtained are shown in FIG. 8. As can be seen, treatment of the cells with either estradiol or spike results in a superimposable decrease in the activity of TRAP, and this effect is blocked by co-treatment with the SERM Raloxifene. These results further demonstrate that spike acts by activating the estrogen receptor.

1. A combination of a Selective Estrogen Receptor Modulator (SERM) and a COVID-19 vaccine.

2. A method for the prevention of COVID-19 in a subject, the method comprising administering the combination according to claim 1 to the subject.

3. A method for the prevention or treatment of side effects of a COVID-19 vaccine in a subject, the method comprising administering a Selective Estrogen Receptor Modulator (SERM) to the subject.

4-5. (canceled)

6. A kit of parts comprising:
a) a COVID-19 vaccine; and
b) a pharmaceutical composition comprising a Selective Estrogen Receptor Modulator (SERM).

7. A combination as claimed in claim 1, wherein said COVID-19 vaccine is a vaccine against COVID-19 that uses the spike protein of SARS-COV-2, a variant thereof or an immunogenic fragment thereof as an antigen.

8. A combination as claimed in claim 7, wherein said COVID-19 vaccine is a mRNA or DNA vaccine.

9. A combination as claimed in claim 1, wherein said Selective Estrogen Receptor Modulator (SERM) is selected from the group consisting of tamoxifen, raloxifene, 4-hydroxy tamoxifen, droloxifene, ospemifene, arzoxifene, toremifene and bazedoxifene.

10. A combination as claimed in claim 1, wherein said subject is a subject diagnosed with an ER-positive breast cancer.

11. A combination as claimed in claim 1, wherein said subject is a subject diagnosed with a pathological condition that alters the physiological haemostatic balance.

12. A method as claimed in claim 2, wherein said COVID-19 vaccine is a vaccine against COVID-19 that uses the spike protein of SARS-COV-2, a variant thereof or an immunogenic fragment thereof as an antigen.

13. A method as claimed in claim 12, wherein said COVID-19 vaccine is a mRNA or DNA vaccine.

14. A method as claimed in claim 2, wherein said Selective Estrogen Receptor Modulator (SERM) is selected from the group consisting of tamoxifen, raloxifene, 4-hydroxy tamoxifen, droloxifene, ospemifene, arzoxifene, toremifene and bazedoxifene.

15. A method as claimed in claim 2, wherein said subject is a subject diagnosed with an ER-positive breast cancer.

16. A method as claimed in claim 2, wherein said subject is a subject diagnosed with a pathological condition that alters the physiological haemostatic balance.

17. A method as claimed in claim 3, wherein said COVID-19 vaccine is a vaccine against COVID-19 that uses the spike protein of SARS-COV-2, a variant thereof or an immunogenic fragment thereof as an antigen.

18. A method as claimed in claim 17, wherein said COVID-19 vaccine is a mRNA or DNA vaccine.

19. A method as claimed in claim 3, wherein said Selective Estrogen Receptor Modulator (SERM) is selected from the group consisting of tamoxifen, raloxifene, 4-hydroxy tamoxifen, droloxifene, ospemifene, arzoxifene, toremifene and bazedoxifene.

20. A method as claimed in claim 3, wherein said subject is a subject diagnosed with an ER-positive breast cancer.

21. A method as claimed in claim 3, wherein said subject is a subject diagnosed with a pathological condition that alters the physiological haemostatic balance.

22. A kit of parts as claimed in claim 6, wherein said COVID-19 vaccine is a vaccine against COVID-19 that uses the spike protein of SARS-COV-2, a variant thereof or an immunogenic fragment thereof as an antigen.

23. A kit of parts as claimed in claim 22, wherein said COVID-19 vaccine is a mRNA or DNA vaccine.

24. A kit of parts as claimed in claim 6, wherein said Selective Estrogen Receptor Modulator (SERM) is selected from the group consisting of tamoxifen, raloxifene, 4-hydroxy tamoxifen, droloxifene, ospemifene, arzoxifene, toremifene and bazedoxifene.
